(19) United States
(12) Patent Application Publication
(10) Pub. No.: US 2022/0152249 A1
(43) Pub. Date: May 19, 2022

(54) A TRANSPARENT PHOTOCATALYTIC COATING FOR IN-SITU GENERATION OF FREE RADICALS COMBATING MICROBES, ODORS OR ORGANIC COMPOUNDS IN VISIBLE LIGHT

(71) Applicant: LEITHA CLEAN TECHNOLOGIES A/S, Skovlunde (DK)

(72) Inventors: Christopher James LÜSCHER, Valby (DK); Diego GARDINI, Frederiksberg (DK)

(21) Appl. No.: 17/440,503

(22) PCT Filed: Mar. 18, 2020
(86) PCT No.: PCT/DK2020/050068

§ 371 Date: Sep. 17, 2021

(30) Foreign Application Priority Data
Mar. 19, 2019 (DK) PA 2019 00338

(51) Int. Cl. A61L 2/22; B01J 21/06; B01J 23/50; B01J 35/02; B01J 35/00; A61L 9/14; A61L 9/01; A61L 2/08; A61L 9/20

(52) U.S. Cl. CPC A61L 2/22; B01J 21/063; B01J 23/50; B01J 35/023; A61L 9/205; B01J 35/026; A61L 9/14; A61L 9/01; A61L 2/088; B01J 35/004

(57) ABSTRACT

A transparent photocatalytic coating for in-situ generation of free radicals combating microbes, odors and organic compounds in visible light is disclosed, featuring a catalytic material comprising a dopant and having particle size distribution suitable for exciton-confinment to accumulatively shift the photocatalytic process into visible light range. Furthermore, the present invention features a method of producing the photocatalytic material described herein. Furthermore, the present invention discloses a method of application of the photocatalytic coating to a surface of a locus. Finally, the present invention features using the photocatalytic coating for removing contaminants and microorganisms at the locus.

### TECHNICAL FIELD

The present invention relates to photocatalytic compositions comprising TiO2, extended to visible light, and, in particular, but not exclusively, to such photocatalytic compositions, intended to reduce the frequency and/or effort of cleaning; and to methods for producing, applying and using such compositions. References will be made herein to photocatalytic compositions which are effective in in-situ generation of free radicals in a broad light range, used in cleaning and combating odors, soils and microorganisms, these being preferred compositions, but descriptions and definitions which follow are applicable also to compositions intended for other purposes.

### Terminology

In the context of present invention, following terms are understood as below:
Exciton: in semiconductors, the term exciton defines a pair made of charged particles (electron, negatively charged and electron hole, positively charged) localized in the material. Alternatively, the term exciton can be used in molecular or atomic physics to describe an excited state of an atom, ion or molecule resulting from the absorption of a defined amount of energy. In this patent, the term exciton is used with both meanings. For example, for TiO2 (a semiconductor) excitons defines the electron-electron hole pairs, for light harvesters (molecules) excitons define the excited electronic states.
Concomitant exciton generation: generation of excitons follows (is concomitant) to the absorption of light. Two mechanisms describe it depending on the nature of the material.
When a semiconductor material such as TiO2 absorbs a photon (light) having an energy greater than its bandgap an exciton can be formed. An electron transitions from the valence band to the conduction band of the material and leaves behind (in the valence band) an electron hole. Valence band and conduction band are energy bands, i.e. energy level ranges. The uppermost energy level of the valence band is separated from the lowermost energy level of the conduction band by an energy gap called bandgap.
In molecules such as methylene blue (presented as an example of light harvesters in the patent), when a photon having an energy corresponding to (or greater than) a transition from the highest occupied molecular orbital (HOMO) to the lowest unoccupied molecular orbital (LUMO) is absorbed, an electron is excited and transitions to the LUMO leaving a hole in the HOMO. Molecular orbitals are energy bands and this mechanisms is called HOMO-LUMO transition.
Exciton-Exciton annihilation: in both semiconductors and molecules, excitons have a recombination lifetime. In semiconductors this means that if electron and electron holes are not spatially separated within a certain amount of time they will recombine cancelling each other (annihilation). In molecules, if the excited electron is not transferred to an energy level of a neighboring material (molecule, ion, atom, crystal) within a certain amount of time, it will return to the lower energy state and the exciton is annihilated. The energy resulting from exciton annihilation can be emitted as phonons (vibrations) or photons (light).
Mineral acid: an acid derived from one or more inorganic compound. Mineral acids dissociate in hydrogen ions and conjugated bases. The examples of mineral acids are: hydrochloric, sulfuric, nitric, perchloric, boric, hydroiodic, hydrobromic and hydrofluoric.
Stabilizer: a chemical compound interacting with suspended nanoparticles having the role to prevent their aggregation. Stabilization can take place in two different ways:
Electrostatic stabilization: the stabilizer provides, enhances or maintains a surface electric charge on the nanoparticles. Nanoparticles bearing a surface charge of same sign (positive or negative) repel each other due to electrostatic forces.
Steric stabilization: the stabilizer molecules bind physically or chemically to the surface of the nanoparticles surrounding them. Steric stabilizers are large molecules and due to their size and extension in space, prevent nanoparticle agglomeration.
Liquid composition: a liquid composition comprising a suspension of TiO2 nanoparticles (insoluble fraction) and a dissolved mineral acid having the role of stabilizing the TiO2 nanoparticles.
Water or pure water: a water with low amount of ionic impurities and conductivity below 20 μS/cm (ISO Type 3, 2 and 1). Demineralized, deionized, distilled, reverse osmosis or milliQ water can be used. Tap water or generally hard water cannot be used as it will lead to nanoparticles aggregation.
Locus: any surface, to which the liquid composition of the present invention can be applied to.
In-situ generation of free radicals: when a semiconductor material such as TiO₂ absorbs a photon (light) having an energy greater than its bandgap, an exciton can be formed. An electron transitions from the valence band to the conduction band of the material and leaves behind (in the valence band) an electron hole. Valence band and conduction band are energy bands, i.e. energy level ranges. The uppermost energy level of the valence band is separated from the lowermost energy level of the conduction band by an energy gap called bandgap. Electrons and holes interact with oxygen species in the surrounding of the semiconductor material to create Reactive Oxygen Species (ROS), which belong to the family of free radicals. Electrons interact with oxygen molecules creating superoxide radicals and holes can interact with water molecules or adsorbed OH⁻ groups to create hydroxyl radicals. ROS can further react to give rise to new radicals, for example superoxide radicals in acidic conditions can react with electrons to generate hydrogen peroxide molecules. Hydrogen peroxide can further interact with either superoxide radicals or electrons to give rise to hydroxyl radicals.
Since free radicals are produced in the close proximity of the semiconductor material (photocatalyst), we call this in-situ generation of free radicals.
Visible light harvesters: a visible light harvester is a substance that can absorb (harvest) photons (light) in the visible range. The visible range is defined as the frequency (or energy) range included between Ultraviolet and Infrared, corresponding to colors visible by the human eye. The role of a light harvester in this context is to absorb photons in the visible range and generate an excited electron. The electron is then transferred to the semiconductor material and can contribute further to the generation of free radicals. In this perspective, visible light harvester extend the optical properties of semiconductors such as TiO₂ that cannot normally absorb light in the visible range.
Synthesis in the presence of reductants: Reductants (also known as reducing agents) are elements or compounds that donate electrons in redox chemical reactions. When a semiconductor oxide such as TiO₂ is synthesized in presence of reductants, a certain amount of Titanium IV atoms (Ti⁴⁺) is reduced to Titanium III atoms (Ti³⁺), a process that causes the loss of oxygen atoms (creation of oxygen vacancies). This reorganization of the semiconductor molecular structure corresponds to a change in electronic and optical properties of the material, in particular the bandgap is narrowed and the absorption of visible light is increased.
Annealing in reducing atmospheres: a reducing atmosphere is a gas composition that includes at least one reducing gas (such as hydrogen). Semiconductors such as TiO₂ can be heated up (annealed) in a reducing atmosphere to produce conversion of Ti⁴⁺ into Ti³⁺ atoms and oxygen vacancies. This is the same as described in the synthesis in the presence of reductants, except that it is done as a treatment after the synthesis of the semiconductor and not during its synthesis.
Capping agent: a capping agent is a substance that binds specifically and stabilize crystalline facets in a crystal. TiO₂, for example, can crystallize in three possible phases: anatase, rutile and brookite. However, it is the anatase phase that shows the highest photocatalytic activity, particularly due to presence of highly reactive anatase {001} crystal facets. These have been shown to produce a more efficient dissociative absorption mechanism in comparison to less reactive {101} facets and reduced recombination rates of photogenerated electron-hole pairs. Engineering anatase TiO₂ to expose a high ratio of {001} facets represents then one of the methods for increasing the production of ROS. Typically, anatase crystals can be found in the shape of a truncated octahedral bipyramid, comprised of eight low reactive {101} facets on the side and two highly reactive {001} facets on the top and bottom. This is the result of a crystal growth in equilibrium conditions, where facets with high energy tends to reduce their area in favor of more thermodynamically-stable ones, minimizing the total surface free energy. However, capping agents such as hydrofluoric acid can be introduced in the synthesis phase, specifically binding to, and stabilizing, highly energetic facets. This results in the synthesis of anatase TiO₂ structures of different shape and aspect ratio (nanosheets), exhibiting a larger ratio of reactive surfaces.
A stabilizer has the role of slowing down or blocking completely the aggregation of semiconductor particles (especially nanoparticles). Contrary to a capping agent its primary role is not to dictate the ratio of exposed specific crystal facets of the semiconductor crystal.

### BACKGROUND

One traditional way of rendering a surface to be self-cleaning and easier to maintain clean is to use antimicrobial coatings that slowly release toxic ingredient, like silver or copper ions; these are difficult to apply and are costly, and an ability to reduce bacterial concentrations to the benign level has a limited lifetime (from hours to days or a few weeks, but not a year and beyond).

Photocatalytic compositions represent another approach to making a contamination-reducing low-bacterial surface. There is a number of photocatalytic compositions known in the prior art, to be applied to various surfaces for in-situ generation of free radicals in order to reduce the frequency of cleaning, and to facilitate the removal of soils deposited on surfaces such as worksurfaces, ceramic tiles, sinks, baths, washbasins, water tanks, toilets, ovens, hobs, carpets, fabrics, floors, painted woodwork, metalwork, laminates, glass surfaces, room door handles, bed rails, taps, sterile packaging, mops, plastics, keyboards, telephones and the like. Making these surfaces contaminant-decomposing and microbe-unfriendly reduces the risk of contamination and infection.

Among the semiconductors, few are suitable for use as photocatalytic materials. The magnitude of the band gap should be chosen accordingly to the light spectrum to be absorbed. Band gaps in the range of 1.2-4 eV are commonly chosen, as covering the visible and near ultraviolet light range. The energetic positions of the band edges should be placed appropriately with respect to the redox potentials of the substances to be mineralized and, equally importantly, with respect to the redox potentials of reactions destroying the semiconductor itself (photocorrosion). Furthermore, the material should be available at reasonable cost, be nontoxic to humans and be capable of being fabricated in a conveniently usable form.

The photoelectrochemical activity of TiO2 was first reported in a pioneering paper by Fujishima and Honda (A. Fujishima and K. Honda, Electrochemical photolysis of water at a semiconductor electrode. Nature (Lond.) 238 (1972) 37-38) and similar processes in nanoparticles were demonstrated a decade later, as well as demonstration of the antimicrobial efficacity of illuminated titanium dioxide nanoparticles was reported. Titanium dioxide has been recognized as one of the few currently known suitable materials for photocatalysis for applications in self-cleaning and antimicrobial coatings, as TiO2 can completely mineralize organic contaminants including microorganisms, producing non toxic byproducts. Further, TiO2 is environmentally benign and inexpensive. Unfortunately, TiO2, which is an excellent photocatalyst under UV light, has very limited capability for visible light absorption.

Silver doping of titania had previously been attempted by A. Vohra et al. (Enhanced photocatalytic inactivation of bacterial spores on surfaces in air. J. Ind. Microbiol. Biotechnol. 32 (2005) 364-370 and idem, Enhanced photocatalytic disinfection of indoor air. Appl. Catal. B 65 (2006) 57-65) reporting that silver doping enhanced the microbicidal efficacy of undoped titania, but the method of doping is not disclosed and the long-term stability of the doped titania material is questionable. Once the cell wall is permeabilized by the photocatalytic activity, metal ions then migrate into the interior of the bacterium. Of course, in such cases the active lifetime of the coating will be limited, because the silver ions will be gradually used up. In summary, there has been a great deal of work on silver doping of titania, with largely disappointing results.

Exciton-exciton annihilation within light harvesters is suppressed by transfer of excited electrons to TiO2, which has high electron affinity. Among the techniques for extending the TiO2 photocatalysis in visible region, mixing with organic dyes is by far the simplest, and it is the basis for dye-sensitized cleaning compositions as disclosed by many, for example, by U.S. Pat. No. 7,438,767 BB (RECKITT BENCKISER GROUP PLC). The residue of such a composition combats soils and undesired microorganisms at the locus. The addition of a monohydric or polyhydric alcohol, preferably having humectant properties, gives benefits in terms of smear avoidance on application and soil removal thereafter. Unfortunately, the dyes are photocatalytically degraded, leading to a short-term benefit. Another examples of organic contaminant is a bacterium with very low levels of light absorption (for example, Staphylococcus aureus) which, in contact with TiO2 (anatase) particles, can harvest visible light and transfer electrons to TiO2 particles, resulting in photocatalytic degradation of said bacterial contaminant (self-degradation catalysed by TiO2). In this mechanism, which is referred to as contaminant activated photocatalysis, the rate of photocatalytic degradation depends on the extent of visible light absorption. This mechanism is utilized to design transparent, contaminant activated photocatalytic coatings for prevention of surface-acquired infections, for example, as disclosed by WO20180123112 A1 (University of Florida Research foundation, INC.).

### SUMMARY

Extension of TiO2 photocatalysis to visible light in this invention disclosure is suggested by combining one or more of the following techniques:

1) creation of defects within the TiO2 crystalline structure, such as oxygen or titanium vacancies or substitutions. Techniques include doping (by for example carbon, nitrogen, sulfur or phosphorous), annealing in reducing atmospheres and synthesis in the presence of reductants;

2) creation of defects at the TiO2 surface. Techniques include surface hydrogenation, plasma treatment and surface amination;

3) combination of visible light harvesters with the TiO2. Techniques include co-synthesis with materials such as gold, copper and quantum dots, and mixing with organic dyes, such as methylene blue, porphyrin and metal-quinoline complexes. The harvester can be the contaminant compound/organism itself, if having any light absorption in visible range.

The three methods differ with respect to the site of visible light absorption and concomitant exciton generation: throughout the modified crystal, at the surface of the modified crystal or in the light harvester.

Our invention discloses further employing any of these methods, most preferably using a dopant, most preferably, the dopant being a silver ion, and using the selfdestruction-catalysing effect of a contaminant/microorganism.

The doping method of this invention suggest a condensation reaction for titania is conducted in presence of a dissolved dopant salt. Very low concentrations of dopant can be used this way to achive a significant effect on TiO2 nanoparticles.

A coating intended to act photocatalytically cannot incorporate a binder, often present in a paint, because the binder would isolate the catalytic particles from microorganisms arriving at the surface, and the binder itself would be photocatalytically degraded. Nanoparticulate is a convenient form of the TiO2 to be applied as a photocatalytic coating. Nanoparticles are very strongly bonded to their substrate, thus lowering the risk of their release into the environment and subsequent inhalation exposure.

Titanium dioxide exists in three polymorphs: anatase, brookite and rutile. Rutile is the stable phase; the other two are metastable. Brookite, the hardest to synthesize and the rarest polymorph, is the least well-known regarding photocatalytic performance and other attributes. The band gap of rutile is 3.0 eV (equivalent to 414 nm; i.e. almost indigo) and it is direct, whereas that of anatase is 3.2 eV (equivalent to 388 nm; i.e. the extreme edge of the violet part of the visible spectrum) and it is indirect. Anatase is, however, a much better photocatalyst than rutile, possibly, due to some differences in the effective masses of the electrons and positive holes, those of anatase being the lightest and, hence, the fastest to migrate after photoexcitation. This enhanced charge separation of anatase is mainly taking place at the {001} facets of the crystals. Anatase TiO2 nanoparticles can be produced in shapes showing an increased area of {001} facets (anisotropic growth), hence increasing the overall photocatalytic coating efficacy. Anatase may also have a more favourable behaviour regarding the adsorption of the reagents essential for the photocatalysed reactions: molecular oxygen and water (both from the air in surrounding atmosphere).

By the novel method of production of the liquid composition comprising TiO2 nanoparticles, disclosed in this application, anatase is being predominantly formed, with an admixture of a fraction of other polymorphs, mainly brookite.

The photocatalytic activity of a nanoparticulate coating can be enhanced by increasing the surface area per weight of photocatalyst. More surface area means that more TiO2 becomes available to interact with the ambient oxygen/water and generates more free radicals. This is achieved by reducing the nominal nanoparticles size. However, reducing the nanoparticle size under a certain value has a secondary unwanted effect of increasing the bandgap of the semiconductor and hence shifting light absorption to shorter wavelength, into the ultraviolet spectrum and outside the visible range. This phenomenom is called exciton quantum confinement and for TiO2 its relevance becomes significant when particle size is reduced below approximately 5 nm (the Bohr radius). So by producing titania particles suspensions with a mean particle distribution of 5-10 nm, the benefits of an high photocatalytic surface area are maximized without loosing significant absorption of visible light due to exciton quantum confinement.

Band-gap narrowing is instead beneficial as it pushes light absorption into the visible range, producing photocatalytic coatings active in the visible light range. This phenomenom is in principle achievable by creating a solid solution of a semiconductor with a narrower band gap than that of pure TiO2. In effect, this can be achieved by doping with sulfur. Doping with nitrogen induces localized states within the bandgap, just above the valence band. This does indeed lead to a red shift of the absorption band edge of anatase, but in rutile a blue shift is occurring because the valence band moves to lower energies as a result of the doping. Unfortunately, the N-doped materials often have poor catalytic activity and, moreover, are often thermally unstable; new states within the bandgap may also serve as electron-hole recombination centres, lowering the quantum yield of photocatalysis. Attempts have been made to overcome these problems by co-doping with other elements, such as molybdenum and vanadium, carbon and carbon nanotubes.

In our invention, we suggest to combine two or more of the 3 main approaches to a red-shift for our TiO2 nanoparticles and wherein the photocatalytic activity per mass of TiO2 nanoparticles is further enhanced by reducing the mean size of said particles up to Bohr radius for exciton quantum confinement for TiO2, which is 4-5 nm and by favouring the growth of specific particle's crystal facets (anisotropic growth), by addition of specific chemicals “capping agents” during synthesis; and wherein said particles can form conglomerates of up to 40 nm in size, still demonstrating the enhancement in photocatalytic activity due to particle size reduction and consequent increase in surface area.

### DETAILED DISCLOSURE

In one aspect, a liquid composition comprising TiO2 nanoparticles is disclosed, where the photocatalytic activity of TiO2 nanoparticles is extended into the visible light by combining one or more of: defects in the crystallinic structure, defects on the surface of nanoparticles, or addition of light harvesters; and further improving the photocatalytic activity of TiO2 nanoparticles by selecting a specific mean size of the particles to be equal to the exciton Bohr radius for semiconductors, being dose to 5 nm for TiO2.

In this aspect, a liquid composition for in-situ generation of free radicals for combating soils, microorganisms and odors at a locus is disclosed, comprising:

a) from 0.01 to 3 percent by weight of TiO2 nanoparticles as a photocatalytic material;

b) from 0.1 to 1 percent by weight of a stabilizer, preferably being a mineral acid, most preferably nitric acid; and

c) the liquid being water;

Characterized in that the photocatalytic activity of TiO2 nanoparticles is extended to be in visible light by:
Created defects within the TiO2 crystalline structure, where created defects within the TiO2 structure are oxygen or titanium vacancies or substitutions, obtained by one or more of the following techniques:
   by doping of TiO2 nanoparticles during their condensation with 0.00001 to 5 percent by weight of one or more dopants selected from the transition metals comprising copper, cobalt, nickel, cromium, manganese, molybdenum, niobium, vanadium, iron, ruthenium, gold, silver, platinum ions and from the non-metals comprising nitrogen, sulfur, carbon, boron, phosphorous, iodine, fluorine ions;
   optionally, by synthesis in the presence of reductants;
   optionally, by annealing in reducing atmospheres.
The TiO2 nanoparticles being 5-10 nm, said particles capable of forming conglomerates of up to 40 nm;
Optionally, combination of visible light harvesters with the TiO2;
Optionally, by created defects at the TiO2 particle surface;

In second aspect, the method of combating the microbes, contaminats and odors using our inventive composition is disclosed:

A method for combating soils, microorganisms and odors at a locus, comprising:
a step of diluting the composition by a factor of 1 (no dilution) to 10 (1 part of composition to 10 parts of pure water);
a step of delivering of said liquid composition to a surface in said locus, comprising an application of said liquid composition, the application process being adapted to deliver most of the TiO2 nanoparticles and only a small fraction of the liquid solvent to the surface; preferably, the application is done using a spraying technique;
a step of drying of said composition at the said surface, and forming a residue or layer of said photocatalytic nanoparticles on said surface, invisible to a human eye.

In a third aspect of the invention, the production method of the compositions according to the first aspect of invention is disclosed:

A method of producing a liquid composition, said production comprising the steps of:

a) mixing of Titania precursor solution with a solvent solution under stirring; preferably, precursor solution is a titanium alkoxide solution, and solvent solution comprises water, a stabilizer and a dopant presursor;

b) purification to remove excess alcohol being formed during the reaction;

c) peptization;

In a forth aspect of the invention, the use of the compositions according to the first aspect of invention at various locuses is disclosed:

The use of a liquid composition as disclosed, applied and produced according to any of the preceding claims, for in-situ generation of free radicals combating soils, microorganisms and odors at a locus, wherein a locus is selected from any indoor or outdoor facility, exemplified by but not limited to an industrial environment, a production facility, a storage house, a vehicle, a home, a hotel, a sport facility, an educational institution, a health care facility, a food or beverage production or serving site, animal farms and other agricultural environments, or elements of these environments, examples being but not restricted to, worksurfaces, ceramic tiles, sinks, baths, washbasins, water tanks, toilets, ovens, hobs, carpets, fabrics, floors, painted woodwork, metalwork, laminates, glass surfaces including windows and mirrors, room door handles, bed rails, taps, sterile packaging, mops, plastics, keyboards, telephones and the like, walls, ceilings, industrial machinery or equipment, shower cubicles, shower curtains, sanitary ware articles, building panels, or kitchen worktops.

### SPECIFIC EXAMPLES OF THE INVENTION

The invention has been described with reference to a number of embodiments and aspects. However, the person skilled in the art may amend such embodiments and aspects while remaining within the scope of the appended patent claims.

Some specific novel and inventive formulations in this scope that have proven efficacy are disclosed in the following embodyments and examples.

### Example 1

A liquid composition comprising a) 0.01-3 wt. % of TiO2 nanoparticles, anatase, average primary size 5-10 nm; b) 0.1-1 wt. % nitric acid; c) 0.00001-0.0025 wt. % AgCl; d) 0-0.1 wt. % isopropanol; e) 95.8975-99.88999 wt. % pure water.

The TiO2 mean particle size of 5-10 nm is equal or right above the Bohr radius. This allows to maximize the TiO2 coating surface area without loosing significant absorption of visible light due to exciton quantum confinement.

Nitric acid is used as a stabilizer to hinder nanoparticle aggregation. The acid works by protonating the surface of the particles and hence giving them a positive surface charge. Charged particles repel each others and do not aggregate. Other acids can be used, such as hydrochloric acid or sulfuric acid. Bases can also be used, and these will give a negative surface charge.

AgCl is used as a source of silver ions. Silver ions act as a dopant, replacing titanium atoms in the TiO2 structure or positioning themselves in interstitials crystal sites in between the atoms of the structure. These modifications change the electronic properties of the semiconductor and allow for absorption of light in the visible range. Other silver salts can be used as a source of silver ions, like silver nitrate AgNO3, silver tetrafluoroborate AgBF4 or silver perchlorate AgClO4. Several other elements can be used instead of silver to provide doping, the most common being copper, cobalt, nickel, cromium, manganese, molybdenum, niobium, vanadium, iron, ruthenium, gold, silver, platinum within transition metals and nitrogen, sulfur, carbon, boron, phosphorous, iodine, fluorine for the non-metals.

Isopropanol is a by-product of the reaction between the titanium precursor (titanium isopropoxide) and water. Depending of the choice of the precursor, other by-products might be present such as butanol (from titanium butoxide) or hydrochloric acid (from titanium tetrachloride).

Water used for the production and in the final product must have a low amount of ionic impurities with conductivity below 20 μS/cm (ISO Type 3, 2 and 1). Demineralized, distilled, reverse osmosis or milliQ water can be used. Tap water or generally hard water cannot be used as it will lead to nanoparticles aggregation.

The photocatalytic activity of TiO2 nanoparticles is further enhanced by favouring the growth of specific particle's crystal facets (anisotropic growth), said favouring is performed using an addition of a capping agent such as hydrofluoric acid HF. In the TiO2 nanoparticle synthesis phase, capping agents specifically bind to and stabilize highly energetic facets such as anatase {001} whose growth would instead be reduced in favour of more thermodynamically stable but less photocatalytically active facets.

### Example 2

Method for delivering a liquid composition combating soils, microorganisms and odors at a locus comprising a) diluting the liquid composition, if necessary, by a factor of 1 (no dilution) to 10 (1 part of composition to 10 parts of pure water);

b) applying composition to a surface, for example, by spraying the composition with an electrostatic spraying gun at a specific distance from the target surface to be coated, so that the visible spraying plume ends 10-20 cm before the target surface; c) let the deposited particles to dry completely, which takes around 2 hours.

### Example 3

A method of producing a liquid composition comprising the steps of a) fast mixing under hgh stirring of 0.1-10 wt. % titanium isopropoxide with a solution of: 88.988-99.88999 wt. % pure water, 0.01-1 wt. % nitric acid and 0.0001-0.002 wt. % AgCl; b) evaporation under vacuum pressure of 1-999 mBar of excess isopropanol being formed during the reaction and c) peptization at a temperature of 30-99 degrees centigrade. These two last steps can be carried out simultaneously for a duration of time which will depend on the initial reagent volumes. Steps b) and c) can be with an advantage performed in a same step, for example, by performing removal of excess alcohol by vacuum evaporation, using a temperature between room temperature and 100 C and absolute pressure between 0.1 mBar and ambient pressure, process time being volume dependent.

### Example 4

The use of a liquid composition as composed, applied and produced according to any of the preceding claims, for in-situ generation of free radicals combating soils, microorganisms and odors at a locus, wherein a locus is selected from any indoor or outdoor facility, exemplified by but not limited to an industrial environment, a production facility, a storage house, a vehicle, a home, a hotel, a sport facility, an educational institution, a health care facility, a food or beverage production or serving site, animal farms and other agricultural environments, or elements of these environments, examples being but not restricted to, a wall, ceiling, floor, window, working surface, industrial machinery or equipment, carpet, mirror, shower cubicle, shower curtain, sanitary ware article, ceramic tile, building panel, water tank or kitchen worktop.

Biocidal active substances are called in situ generated active substances if they are generated from one or more precursors at the place of use. In our invention, the TiO2 particles are catalysing the formation of free radicals from the ambient air or water, depending on the application site. Coated on the inside surface of a fish tank, as an example, our photocatalysator wil generate in-situ free radicals out of water molecules and dissolved gases and salts present in water.

### Example 5

Tests of efficacy of the liquid composition of our invention on patogens are summarized below:

| | | | [symbol] | [symbol] | [symbol] | |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | | [symbol] | [symbol] | [symbol] |
| | [symbol] | [symbol] | | [symbol] | [symbol] | [symbol] |
| [symbol] | [symbol] | [symbol] | | | [symbol] | [symbol] |
| [symbol] | [symbol] | [symbol] | | | [symbol] | [symbol] |
| [symbol] | [symbol] | [symbol] | | | [symbol] | [symbol] |

[symbol]

[symbol]

indicates data missing or illegible when filed

European Standards (abbreviated ENs owing to the more literal translation from French/German as European Norms) are technical standards drafted and maintained by CEN (European Committee for Standardization). CENELEC (European Committee for Electrotechnical Standardization) and ETSI (European Telecommunications Standards Institute).
1 log reduction=90% reduction
2 log reduction=99% reduction
3 log reduction=99.9% reduction
4 log reduction=99.99% reduction
5 log reduction=99.999% reduction
6 log reduction=99.9999% reduction

### Example 1 Embodyments

1. Composition comprising a) 0.01 wt. % of TiO2 nanoparticles, anatase; b) 0.1 wt. % nitric acid; c) 0,00001 wt. % AgCl; d) traces of isopropanol; e) balance is water.
2. Composition comprising a) 0.01 wt. % of TiO2 nanoparticles, anatase; b) 0.1 wt. % nitric acid; c) 0,0001 wt. % AgCl; d) traces of isopropanol; e) balance is water.
3. Composition comprising a) 0.01 wt. % of TiO2 nanoparticles, anatase; b) 0.1 wt. % nitric acid; c) 0,001 wt. % AgCl; d) traces of isopropanol; e) balance is water.
4. Composition comprising a) 0.01 wt. % of TiO2 nanoparticles, anatase; b) 0.1 wt. % nitric acid; c) 0.0025 wt. % AgCl; d) traces of isopropanol; e) balance is water.
5. Composition comprising a) 0.01 wt. % of TiO2 nanoparticles, anatase; b) 0.3 wt. % nitric acid; c) 0,00001 wt. % AgCl; d) traces of isopropanol; e) balance is water.
6. Composition comprising a) 0.01 wt. % of TiO2 nanoparticles, anatase; b) 0.3 wt. % nitric acid; c) 0,0001 wt. % AgCl; d) traces of isopropanol; e) balance is water.
7. Composition comprising a) 0.01 wt. % of TiO2 nanoparticles, anatase; b) 0.3 wt. % nitric acid; c) 0,001 wt. % AgCl; d) traces of isopropanol; e) balance is water.
8. Composition comprising a) 0.01 wt. % of TiO2 nanoparticles, anatase; b) 0.3 wt. % nitric acid; c) 0.0025 wt. % AgCl; d) traces of isopropanol; e) balance is water.
9. Composition comprising a) 0.01 wt. % of TiO2 nanoparticles, anatase; b) 0.7 wt. % nitric acid; c) 0,00001 wt. % AgCl; d) traces of isopropanol; e) balance is water.
10. Composition comprising a) 0.01 wt. % of TiO2 nanoparticles, anatase; b) 0.7 wt. % nitric acid; c) 0,0001 wt. % AgCl; d) traces of isopropanol; e) balance is water.
11. Composition comprising a) 0.01 wt. % of TiO2 nanoparticles, anatase; b) 0.7 wt. % nitric acid; c) 0,001 wt. % AgCl; d) traces of isopropanol; e) balance is water.
12. Composition comprising a) 0.01 wt. % of TiO2 nanoparticles, anatase; b) 0.7 wt. % nitric acid; c) 0.0025 wt. % AgCl; d) traces of isopropanol; e) balance is water.
13. Composition comprising a) 0.01 wt. % of TiO2 nanoparticles, anatase; b) 1 wt. % nitric acid; c) 0,00001 wt. % AgCl; d) traces of isopropanol; e) balance is water.
14. Composition comprising a) 0.01 wt. % of TiO2 nanoparticles, anatase; b) 1 wt. % nitric acid; c) 0,0001 wt. % AgCl; d) traces of isopropanol; e) balance is water.
15. Composition comprising a) 0.01 wt. % of TiO2 nanoparticles, anatase; b) 1 wt. % nitric acid; c) 0,001 wt. % AgCl; d) traces of isopropanol; e) balance is water.
16. Composition comprising a) 0.01 wt. % of TiO2 nanoparticles, anatase; b) 1 wt. % nitric acid; c) 0.0025 wt. % AgCl; d) traces of isopropanol; e) balance is water.
17. Composition comprising a) 0.1 wt. % of TiO2 nanoparticles, anatase; b) 0.1 wt. % nitric acid; c) 0,00001 wt. % AgCl; d) traces of isopropanol; e) balance is water.
18. Composition comprising a) 0.1 wt. % of TiO2 nanoparticles, anatase; b) 0.1 wt. % nitric acid; c) 0 wt. % AgCl; d) traces of isopropanol; e) balance is water.
19. Composition comprising a) 0.1 wt. % of TiO2 nanoparticles, anatase; b) 0.1 wt. % nitric acid; c) 0 wt. % AgCl; d) traces of isopropanol; e) balance is water.
20. Composition comprising a) 0.1 wt. % of TiO2 nanoparticles, anatase; b) 0.1 wt. % nitric acid; c) 0 wt. % AgCl; d) traces of isopropanol; e) balance is water.
21. Composition comprising a) 0.1 wt. % of TiO2 nanoparticles, anatase; b) 0.3 wt. % nitric acid; c) 0,00001 wt. % AgCl; d) traces of isopropanol; e) balance is water.
22. Composition comprising a) 0.1 wt. % of TiO2 nanoparticles, anatase; b) 0.3 wt. % nitric acid; c) 0,0001 wt. % AgCl; d) traces of isopropanol; e) balance is water.
23. Composition comprising a) 0.1 wt. % of TiO2 nanoparticles, anatase; b) 0.3 wt. % nitric acid; c) 0,001 wt. % AgCl; d) traces of isopropanol; e) balance is water.
24. Composition comprising a) 0.1 wt. % of TiO2 nanoparticles, anatase; b) 0.3 wt. % nitric acid; c) 0.0025 wt. % AgCl; d) traces of isopropanol; e) balance is water.
25. Composition comprising a) 0.1 wt. % of TiO2 nanoparticles, anatase; b) 0.7 wt. % nitric acid; c) 0,00001 wt. % AgCl; d) traces of isopropanol; e) balance is water.
26. Composition comprising a) 0.1 wt. % of TiO2 nanoparticles, anatase; b) 0.7 wt. % nitric acid; c) 0,0001 wt. % AgCl; d) traces of isopropanol; e) balance is water.
27. Composition comprising a) 0.1 wt. % of TiO2 nanoparticles, anatase; b) 0.7 wt. % nitric acid; c) 0,001 wt. % AgCl; d) traces of isopropanol; e) balance is water.
28. Composition comprising a) 0.1 wt. % of TiO2 nanoparticles, anatase; b) 0.7 wt. % nitric acid; c) 0.0025 wt. % AgCl; d) traces of isopropanol; e) balance is water.
29. Composition comprising a) 0.1 wt. % of TiO2 nanoparticles, anatase; b) 1 wt. % nitric acid; c) 0,00001 wt. % AgCl; d) traces of isopropanol; e) balance is water.
30. Composition comprising a) 0.1 wt. % of TiO2 nanoparticles, anatase; b) 1 wt. % nitric acid; c) 0,0001 wt. % AgCl; d) traces of isopropanol; e) balance is water.
31. Composition comprising a) 0.1 wt. % of TiO2 nanoparticles, anatase; b) 1 wt. % nitric acid; c) 0,001 wt. % AgCl; d) traces of isopropanol; e) balance is water.
32. Composition comprising a) 0.1 wt. % of TiO2 nanoparticles, anatase; b) 1 wt. % nitric acid; c) 0.0025 wt. % AgCl; d) traces of isopropanol; e) balance is water.
33. Composition comprising a) 1.5 wt. % of TiO2 nanoparticles, anatase; b) 0.1 wt. % nitric acid; c) 0,00001 wt. % AgCl; d) traces of isopropanol; e) balance is water.
34. Composition comprising a) 1.5 wt. % of TiO2 nanoparticles, anatase; b) 0.1 wt. % nitric acid; c) 0 wt. % AgCl; d) traces of isopropanol; e) balance is water.
35. Composition comprising a) 1.5 wt. % of TiO2 nanoparticles, anatase; b) 0.1 wt. % nitric acid; c) 0 wt. % AgCl; d) traces of isopropanol; e) balance is water.
36. Composition comprising a) 1.5 wt. % of TiO2 nanoparticles, anatase; b) 0.1 wt. % nitric acid; c) 0 wt. % AgCl; d) traces of isopropanol; e) balance is water.
37. Composition comprising a) 1.5 wt. % of TiO2 nanoparticles, anatase; b) 0.3 wt. % nitric acid; c) 0,00001 wt. % AgCl; d) traces of isopropanol; e) balance is water.
38. Composition comprising a) 1.5 wt. % of TiO2 nanoparticles, anatase; b) 0.3 wt. % nitric acid; c) 0,0001 wt. % AgCl; d) traces of isopropanol; e) balance is water.
39. Composition comprising a) 1.5 wt. % of TiO2 nanoparticles, anatase; b) 0.3 wt. % nitric acid; c) 0,001 wt. % AgCl; d) traces of isopropanol; e) balance is water.
40. Composition comprising a) 1.5 wt. % of TiO2 nanoparticles, anatase; b) 0.3 wt. % nitric acid; c) 0.0025 wt. % AgCl; d) traces of isopropanol; e) balance is water.
41. Composition comprising a) 1.5 wt. % of TiO2 nanoparticles, anatase; b) 0.7 wt. % nitric acid; c) 0,00001 wt. % AgCl; d) traces of isopropanol; e) balance is water.
42. Composition comprising a) 1.5 wt. % of TiO2 nanoparticles, anatase; b) 0.7 wt. % nitric acid; c) 0,0001 wt. % AgCl; d) traces of isopropanol; e) balance is water.
43. Composition comprising a) 1.5 wt. % of TiO2 nanoparticles, anatase; b) 0.7 wt. % nitric acid; c) 0,001 wt. % AgCl; d) traces of isopropanol; e) balance is water.
44. Composition comprising a) 1.5 wt. % of TiO2 nanoparticles, anatase; b) 0.7 wt. % nitric acid; c) 0.0025 wt. % AgCl; d) traces of isopropanol; e) balance is water.
45. Composition comprising a) 1.5 wt. % of TiO2 nanoparticles, anatase; b) 1 wt. % nitric acid; c) 0,00001 wt. % AgCl; d) traces of isopropanol; e) balance is water.
46. Composition comprising a) 1.5 wt. % of TiO2 nanoparticles, anatase; b) 1 wt. % nitric acid; c) 0,0001 wt. % AgCl; d) traces of isopropanol; e) balance is water.
47. Composition comprising a) 1.5 wt. % of TiO2 nanoparticles, anatase; b) 1 wt. % nitric acid; c) 0,001 wt. % AgCl; d) traces of isopropanol; e) balance is water.
48. Composition comprising a) 1.5 wt. % of TiO2 nanoparticles, anatase; b) 1 wt. % nitric acid; c) 0.0025 wt. % AgCl; d) traces of isopropanol; e) balance is water.
49. Composition comprising a) 3 wt. % of TiO2 nanoparticles, anatase; b) 0.1 wt. % nitric acid; c) 0,00001 wt. % AgCl; d) traces of isopropanol; e) balance is water.
50. Composition comprising a) 3 wt. % of TiO2 nanoparticles, anatase; b) 0.1 wt. % nitric acid; c) 0 wt. % AgCl; d) traces of isopropanol; e) balance is water.
51. Composition comprising a) 3 wt. % of TiO2 nanoparticles, anatase; b) 0.1 wt. % nitric acid; c) 0 wt. % AgCl; d) traces of isopropanol; e) balance is water.
52. Composition comprising a) 3 wt. % of TiO2 nanoparticles, anatase; b) 0.1 wt. % nitric acid; c) 0 wt. % AgCl; d) traces of isopropanol; e) balance is water.
53. Composition comprising a) 3 wt. % of TiO2 nanoparticles, anatase; b) 0.3 wt. % nitric acid; c) 0,00001 wt. % AgCl; d) traces of isopropanol; e) balance is water.
54. Composition comprising a) 3 wt. % of TiO2 nanoparticles, anatase; b) 0.3 wt. % nitric acid; c) 0,0001 wt. % AgCl; d) traces of isopropanol; e) balance is water.
55. Composition comprising a) 3 wt. % of TiO2 nanoparticles, anatase; b) 0.3 wt. % nitric acid; c) 0,001 wt. % AgCl; d) traces of isopropanol; e) balance is water.
56. Composition comprising a) 3 wt. % of TiO2 nanoparticles, anatase; b) 0.3 wt. % nitric acid; c) 0.0025 wt. % AgCl; d) traces of isopropanol; e) balance is water.
57. Composition comprising a) 3 wt. % of TiO2 nanoparticles, anatase; b) 0.7 wt. % nitric acid; c) 0,00001 wt. % AgCl; d) traces of isopropanol; e) balance is water.
58. Composition comprising a) 3 wt. % of TiO2 nanoparticles, anatase; b) 0.7 wt. % nitric acid; c) 0,0001 wt. % AgCl; d) traces of isopropanol; e) balance is water.
59. Composition comprising a) 3 wt. % of TiO2 nanoparticles, anatase; b) 0.7 wt. % nitric acid; c) 0,001 wt. % AgCl; d) traces of isopropanol; e) balance is water.
60. Composition comprising a) 3 wt. % of TiO2 nanoparticles, anatase; b) 0.7 wt. % nitric acid; c) 0.0025 wt. % AgCl; d) traces of isopropanol; e) balance is water.
61. Composition comprising a) 3 wt. % of TiO2 nanoparticles, anatase; b) 1 wt. % nitric acid; c) 0,00001 wt. % AgCl; d) traces of isopropanol; e) balance is water.
62. Composition comprising a) 3 wt. % of TiO2 nanoparticles, anatase; b) 1 wt. % nitric acid; c) 0,0001 wt. % AgCl; d) traces of isopropanol; e) balance is water.
63. Composition comprising a) 3 wt. % of TiO2 nanoparticles, anatase; b) 1 wt. % nitric acid; c) 0,001 wt. % AgCl; d) traces of isopropanol; e) balance is water.
64. Composition comprising a) 3 wt. % of TiO2 nanoparticles, anatase; b) 1 wt. % nitric acid; c) 0.0025 wt. % AgCl; d) traces of isopropanol; e) balance is water.

1-10. (canceled)

11. A liquid composition for in-situ generation of free radicals for combating soils, microorganisms and odors at a locus, comprising:
a) from 0.01 to 3 percent by weight of TiO₂ nanoparticles as a photocatalytic material;
b) from 0.1 to 1 percent by weight of a mineral acid as stabilizer, and
c) the liquid being water;
wherein the photocatalytic activity of TiO₂ nanoparticles is extended to be in visible light by:
Created defects within the TiO₂ crystalline structure, wherein said created defects within the TiO₂ structure are oxygen or titanium vacancies or substitutions obtained by one or more of the following techniques:
by doping of TiO₂ nanoparticles during their condensation with 0.00001 to 5 percent by weight of one or more dopants comprising (i) one or more of copper, cobalt, nickel, chromium, manganese, molybdenum, niobium, vanadium, iron, ruthenium, gold, silver, platinum ions, and (ii) one or more of nitrogen, sulfur, carbon, boron, phosphorous, iodine, and fluorine ions;
optionally, by synthesis in the presence of reductants;
optionally, by annealing in reducing atmospheres.
The TiO₂ nanoparticles being 5-10 nm, said particles capable of forming conglomerates of up to 40 nm;
Optionally, combination of visible light harvesters with the TiO₂;
Optionally, by created defects at the TiO₂ particle surface.

12. The composition according to claim 11, the composition comprising 2 percent by weight of TiO₂ particles.

13. The composition according to claim 11, wherein the dopant is silver ions and the concentration of silver dopant is 0.0025 percent by weight.

14. The composition according to claim 11, wherein the combination of visible light harvesters with the TiO₂ is obtained by one or more of the following techniques:
by the contaminant compound/microorganism itself, having an absorption in visual light;
optionally, by co-synthesis of TiO₂ nanoparticles;
optionally, by mixing with organic dyes.

15. The composition according to claim 11, wherein the created defects at the TiO₂ particle surface are obtained by one or more of the following techniques:
surface chemical modifications; or
by plasma treatment.

16. The composition according to claim 11, where the photocatalytic activity of TiO₂ nanoparticles is further enhanced by promoting growth of a specific particle's crystal facets, with said promoting being effected by addition of a capping agent.

17. A method for combating soils, microorganisms and odors at a locus, using the composition of any of the preceding claims, the method comprising:
optionally diluting the composition by up to a factor of 10;
delivering of said liquid composition to a surface in said locus so as to deliver most of the TiO₂ nanoparticles and a fraction of the liquid solvent to the surface; and
drying of said composition at the said surface.

18. The method of producing a liquid composition according to claim 11, said method comprising the steps of:
a) mixing of a titania precursor solution with a solvent solution under stirring; the precursor solution optionally being a titanium alkoxide solution, and the solvent solution optionally comprising water, a stabilizer and a dopant precursor;
b) purification to remove excess alcohol being formed during the reaction;
c) peptization.

19. The method according to claim 18, wherein b) and c) are carried out simultaneously.

20. The use of a liquid composition according to claim 11 for in-situ generation of free radicals combating soils, microorganisms and odors at a locus, wherein a locus is selected from any indoor or outdoor facility, including an industrial environment, a production facility, a storage house, a vehicle, a home, a hotel, a sport facility, an educational institution, a health care facility, a food or beverage production or serving site, an animal farm, and agricultural environments, or an elements of the forgoing.
